(19) United States
(12) Patent Application Publication
(10) Pub. No.: US 2026/0000442 A1
(43) Pub. Date: Jan. 1, 2026

(54) ENDOSCOPIC TREATMENT INSTRUMENT

(71) Applicant: OLYMPUS MEDICAL SYSTEMS CORP., Tokyo (JP)

(72) Inventors: Yusuke SHIOTA, Machida-shi (JP); Yuta Muyari, Hino-shi (JP); Shunsuke Motosugi, Hachioji-shi (JP)

(21) Appl. No.: 19/038,353

(22) Filed: Jan. 27, 2025

Related U.S. Application Data
(63) Continuation of application No. PCT/JP2024/023497, filed on Jun. 28, 2024.

(51) Int. Cl. A61B 18/14; A61B 1/00; A61B 1/005; A61B 1/018; A61B 18/00; A61B 90/00

(52) U.S. Cl. CPC A61B 18/14; A61B 1/00119; A61B 1/005; A61B 1/018; A61B 90/03; A61B 2018/00083; A61B 2018/00101; A61B 2018/00178; A61B 2018/00595; A61B 2018/00982; A61B 2090/036; A61B 2218/007

(57) ABSTRACT

An endoscopic treatment instrument includes a sheath including a first tube and a distal end member, the distal end member fixed to a distal end of the first tube, the distal end member including a suction hole, an electrode protruding from a distal end of the sheath, a second tube fixed to the distal end member, a conduit located between an inner surface of the second tube and an outer surface of the first tube, the conduit communicating with the suction hole. A proximal end of the conduit is located between a distal end and a proximal end of the first tube.

### CROSS-REFERENCE TO RELATED APPLICATIONS

The present application is a continuation application based on PCT Patent Application No. PCT/JP2024/023497, filed on Jun. 28, 2024, the entire content of which is hereby incorporated by reference.

### FIELD

The present disclosure relates to an endoscopic treatment instrument.

### BACKGROUND

Endoscopic treatment instruments for incision and dissection such as high-frequency knives and endoscopic treatment instruments for hemostasis are used in endoscopic therapy such as endoscopic submucosal dissection (ESD).

When hemostasis is performed using an endoscopic treatment instrument, a practitioner performs a water supply process to identify a hemostatic location. When the hemostasis is performed after the hemostatic location is identified, it is good to eliminate a liquid serving as an electrolyte in the vicinity of the hemostatic location. Therefore, the practitioner performs a treatment process of suctioning a liquid around the tissue of a hemostasis target before hemostasis. As described in United States Patent Application, Publication No. 2007/0038213 (hereinafter referred to as Patent Document 1) and Japanese Unexamined Patent Application, First Publication No. 2009-233269 (hereinafter referred to as Patent Document 2), an endoscopic treatment instrument has a suction function.

### SUMMARY

Because an endoscopic treatment instrument described in Patent Document 1 and Patent Document 2 has a suction conduit on the entire length of the sheath, a conduit diameter needs to be increased to improve the suction efficiency. In this case, an outer diameter of the sheath can be also increased across the entire length. If the outer diameter of the sheath increases across the entire length, the suction performance of the endoscope is likely to be lowered.

The present disclosure provides an endoscopic treatment instrument that can perform a suction process from a distal end of a sheath without significant deterioration in the suction performance of an endoscope.

According to a first aspect of the present disclosure, there is provided an endoscopic treatment instrument comprising: a sheath including a first tube and a distal end member, the distal end member fixed to a distal end of the first tube, the distal end member including a suction hole; an electrode protruding from a distal end of the sheath; a second tube fixed to the distal end member; and a conduit located between an inner surface of the second tube and an outer surface of the first tube, the conduit communicating with the suction hole, wherein a proximal end of the conduit is located between a distal end and a proximal end of the first tube.

According to the present disclosure, an endoscopic treatment instrument can perform a suction process from a distal end of a sheath without significant deterioration in the suction performance of an endoscope.

### BRIEF DESCRIPTION OF DRAWINGS

FIG. 1 is an overall view of an endoscopic treatment system according to a first embodiment.

FIG. 2 is an overall view showing a treatment instrument of the endoscopic treatment system.

FIG. 3 is a perspective view of a distal end opening of a channel of an endoscope of the endoscopic treatment system.

FIG. 4 is a perspective view of a distal end portion of the treatment instrument.

FIG. 5 is a view showing the distal end portion of the treatment instrument in which a distal end of a knife is at a second position.

FIG. 6 is a cross-sectional view along line X1-X1 of FIG. 5.

FIG. 7 is a cross-sectional view along line X2-X2 of FIG. 5.

FIG. 8 is a view showing the distal end portion of the treatment instrument in which a distal end of the knife is located at a first position.

FIG. 9 is a cross-sectional view of the distal end portion of the treatment instrument.

FIG. 10 is a view showing a modified example of a suction hole.

FIG. 11 is a view showing another modified example of the suction hole.

FIG. 12 is a view showing a modified example of a knife.

FIG. 13 is a cross-sectional view of a modified example of the treatment instrument.

FIG. 14 is a cross-sectional view of a modified example of the channel.

FIG. 15 is a perspective view of a distal end portion of a treatment instrument according to a second embodiment.

FIG. 16 is a cross-sectional view of the distal end portion of the treatment instrument in which a distal end of a knife is at a second position.

FIG. 17 is a cross-sectional view of the distal end portion of the treatment instrument in which the distal end of the knife is at a first position.

FIG. 18 is a perspective view of a distal end portion of a treatment instrument according to a third embodiment.

FIG. 19 is a cross-sectional view of the distal end portion of the treatment instrument.

### DETAILED DESCRIPTION

### First Embodiment

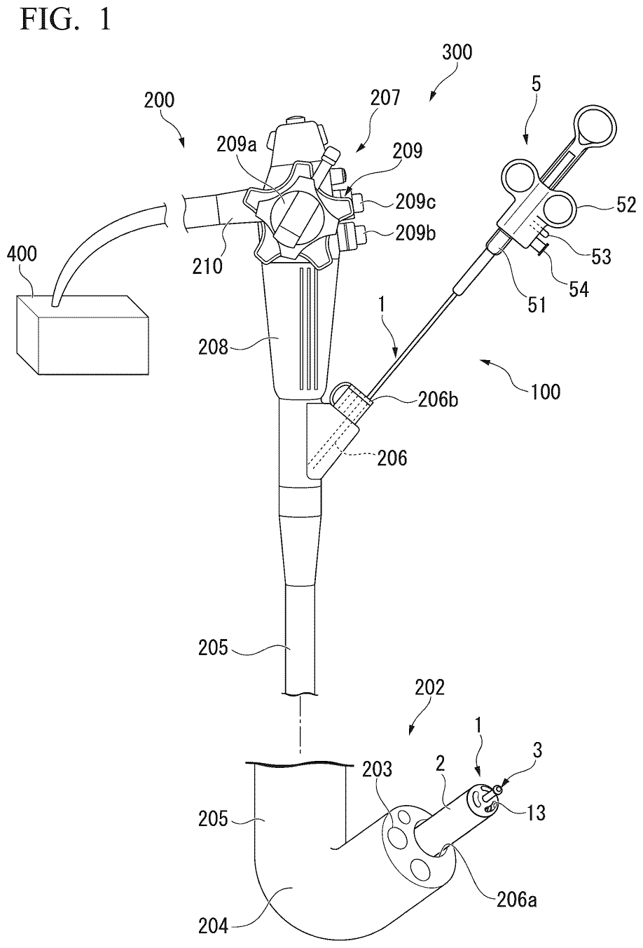

An endoscopic treatment system 300 according to a first embodiment will be described with reference to FIGS. 1 to 9. FIG. 1 is an overall view of the endoscopic treatment system 300 according to the present embodiment.

### [Endoscopic Treatment System 300]

As shown in FIG. 1, the endoscopic treatment system 300 includes an endoscope 200 and a treatment instrument 100. The treatment instrument 100 is used in a state in which it is inserted into the endoscope 200.

### [Endoscope 200]

The endoscope 200 is a flexible endoscope and includes an insertion portion 202 inserted into a body from a distal end and a manipulation portion 207 attached to a proximal end of the insertion portion 202.

The insertion portion 202 includes an imaging portion 203, a bending portion 204, and a flexible portion 205. From the distal end of the insertion portion 202, the imaging portion 203, the bending portion 204, and the flexible portion 205 are arranged in that order. Inside the insertion portion 202, a channel 206 for inserting the treatment instrument 100 is provided. A distal end opening 206a of the channel 206 is provided at the distal end of the insertion portion 202.

The imaging portion 203 includes, for example, an imaging element such as a charge-coupled device (CCD) or a complementary metal-oxide semiconductor (CMOS), and can image a portion serving as a treatment target. The imaging portion 203 can image a knife 3 of the treatment instrument 100 in a state in which the treatment instrument 100 protrudes from the distal end opening 206a of the channel 206.

The bending portion 204 is bent according to a manipulation of a manipulator on the manipulation portion 207. The flexible portion 205 is a tubular part having flexibility.

The manipulation portion 207 is connected to the flexible portion 205. The manipulation portion 207 includes a grip 208, an input portion 209, a proximal end opening (a forceps mouth) 206b of the channel 206, and a universal cord 210. The grip 208 is a part that is gripped by the manipulator.

The input portion 209 includes an angle knob 209a, an air supply/water supply button 209b, and a suction button 209c. The angle knob 209a receives a manipulation input for bending the bending portion 204. The air supply/water supply button 209b is a push button for inputting an air supply/water supply manipulation from the distal end opening 206a. The suction button 209c is a push button for inputting a suction manipulation from the distal end opening 206a and suction in the channel is started by pressing the suction button 209c.

The universal cord 210 externally outputs an image captured by the imaging portion 203. The universal cord 210 is connected to a display device such as a liquid crystal display via an image processing device including a processor or the like. In addition, the endoscope 200 is connected to a suction pump 400 via the universal cord 210, and suction in the channel 206 is started by pressing the suction button 209c.

### [Treatment Instrument 100]

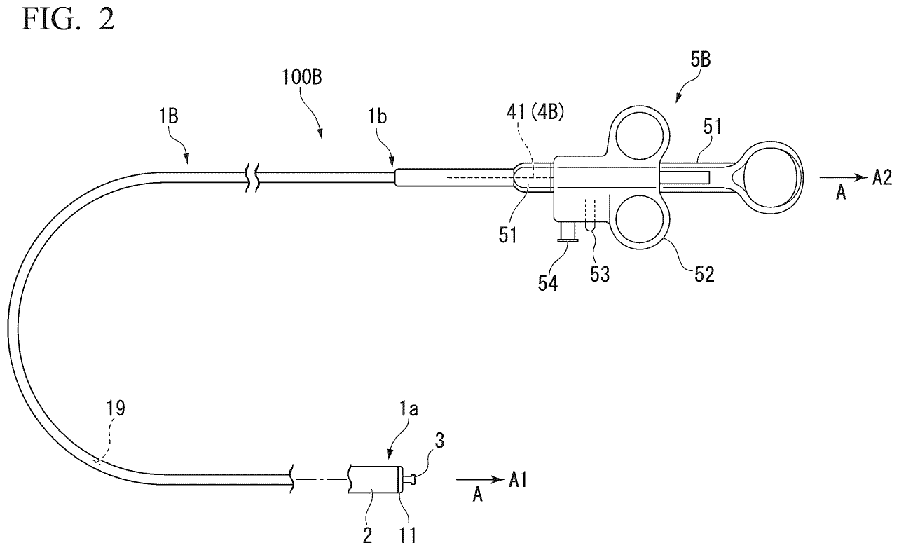

FIG. 2 is an overall view showing the treatment instrument 100.

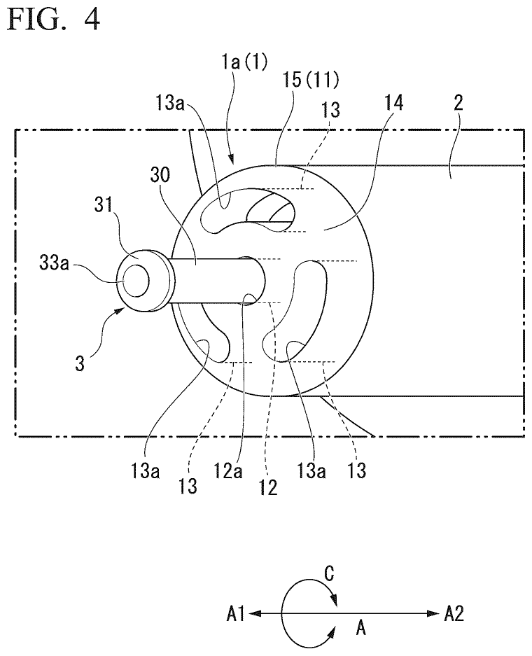

The treatment instrument (an endoscopic treatment instrument or a high-frequency treatment instrument) 100 includes a sheath 1, a suction tube (second tube) 2, a knife 3, a manipulation wire (wire) 4 (see FIG. 4), and a manipulation portion 5. In the following description, in a longitudinal direction A of the treatment instrument 100, a side inserted into a patient's body is referred to as a “distal end side (a distal side) A1” and the manipulation portion 5 side is referred to as a “proximal end side (a proximal side) A2.”

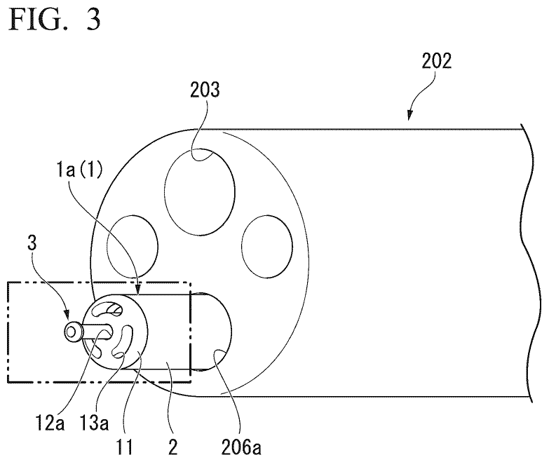

FIG. 3 is a perspective view of the distal end opening 206a of the channel 206. The sheath 1 is a long tubular member extending from a distal end portion 1a to a proximal end portion 1b in a longitudinal direction A along a longitudinal shaft. The sheath 1 can be inserted into the channel 206 of the endoscope 200 and can advance and retract in the channel 206. In a state in which the sheath 1 is inserted into the channel 206, the distal end portion 1a of the sheath 1 can protrude or retract from the distal end opening 206a of the channel 206.

FIG. 4 is a perspective view of a distal end portion of the treatment instrument 100.

The sheath 1 includes an outer tube (first tube) 10 extending in a longitudinal direction A and a distal end member 11 provided at the distal end of the outer tube 10. The sheath 1 may be formed by integrally molding the outer tube 10 and the distal end member 11 or may be made of the same material.

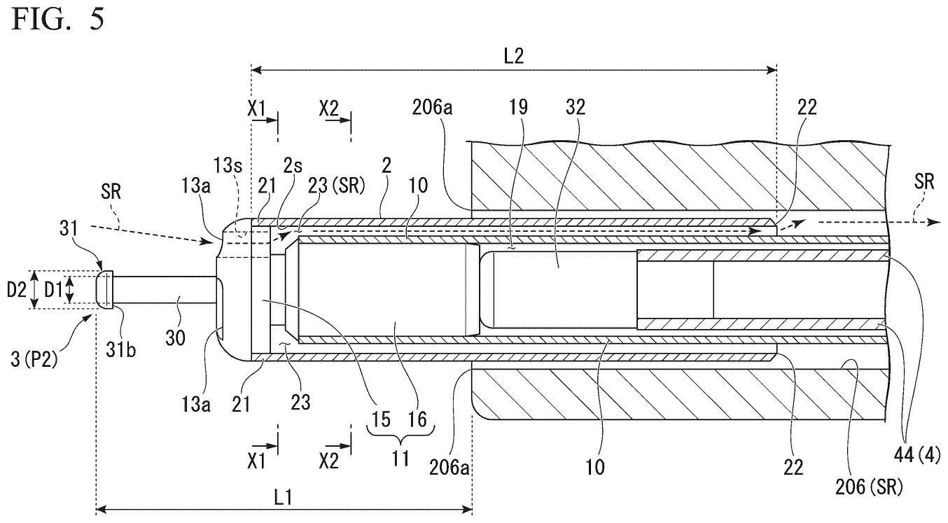

FIG. 5 is a view showing the distal end portion of the treatment instrument 100 in which the distal end of the knife 3 is at a second position P2.

The sheath 1 is inserted into an internal space (conduit) 23 of the suction tube 2 (hereinafter also referred to as a “suction conduit 23”). A part of the knife 3 is inserted into the internal space 19 of the sheath 1. That is, an outer diameter of the sheath 1 is smaller than an outer diameter of the suction tube 2 and larger than an outer diameter of the knife 3.

The outer tube 10 is a long tubular member having flexibility and insulation. The outer tube 10 is formed of, for example, resin. In the present embodiment, the internal space 19 of the outer tube 10 is a space into which a part of the knife 3 can be inserted.

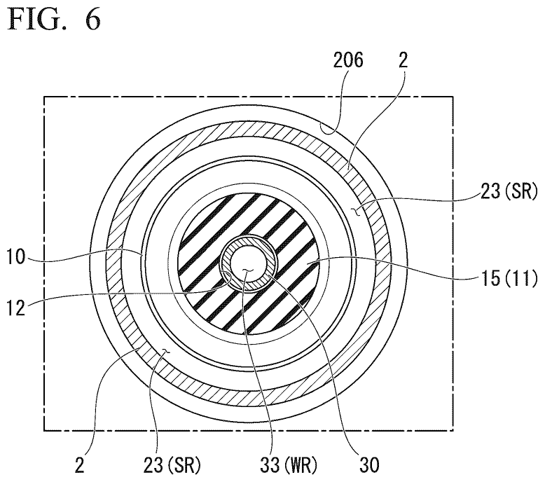

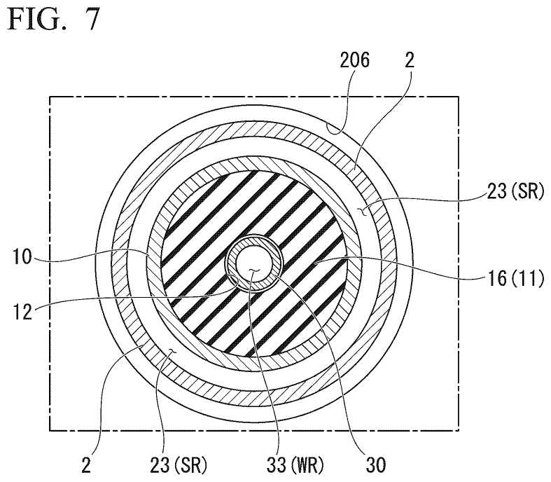

FIG. 6 is a cross-sectional view along line X1-X1 of FIG. 5. FIG. 7 is a cross-sectional view along line X2-X2 of FIG. 5. The distal end member 11 is formed in a cylinder-like shape. In addition, the “cylinder-like shape” includes an exact cylindrical shape and an approximately cylindrical shape. The distal end member 11 can be formed of an insulating material such as resin. A length from the distal end to the proximal end of the distal end member 11 is, for example, 10 mm to 20 mm. As shown in FIG. 4, a through-hole 12 and a suction hole 13 are formed in the distal end member 11. There is no direct communication between the through-hole 12 and the suction hole 13. The distal end member 11 has a first portion (insulator) 15 and a second portion (third tube) 16.

The first portion 15 is provided on the distal end side A1 of the distal end member 11 and is formed in a disk-like shape. In addition, the “disk-like shape” includes an exact disk shape and an approximately disk shape. At least a part of the first portion 15 protrudes from the outer tube 10 to the distal end side A1. As shown in FIG. 4, the first portion 15 has a distal end surface 14 on the distal end side A1. At least a part of the first portion 15 is inserted into the suction tube 2 and the distal end opening 21 of the suction tube 2 is fitted and fixed. The first portion 15 protrudes from the distal end of the suction tube 2 to the distal end side A1. The outer diameter of the first portion 15 is larger than the outer diameter of the outer tube 10.

The second portion 16 is provided on the proximal end side A2 of the distal end member 11 and is formed in a cylinder-like shape. In addition, the “cylinder-like shape” includes an exact disk shape and an approximately cylindrical shape. The second portion 16 is provided on the proximal end side A2 of the first portion 15. The second portion 16 is inserted into the outer tube 10 and the distal end opening of the outer tube 10 is fitted and fixed. For example, the second portion 16 of the distal end member 11 and the outer tube 10 may be joined by press-fitting the outer tube 10 to a tapping screw provided on the distal end member 11. Moreover, the second portion 16 of the distal end member 11 and the outer tube 10 may be joined by an adhesive.

The through-hole 12 is provided in the first portion 15 and the second portion 16 of the distal end member 11 and is a hole passing through the distal end member 11 in the longitudinal direction A. The distal end of the through-hole 12 communicates with the first opening 12a formed in the distal end surface 14 of the distal end member 11. The proximal end of the through-hole 12 communicates with the internal space 19 of the outer tube 10.

The suction hole 13 is formed in the first portion 15 of the distal end member 11 and passes through the first portion 15 of the distal end member 11 in the longitudinal direction A and forms at least a part of a suction channel SR. Three suction holes 13 are formed in the first portion 15 of the distal end member 11. The three suction holes 13 communicate with the three distal end openings 13a formed in the distal end surface 14 of the distal end member 11. The proximal end of the suction hole 13 communicates with the suction conduit 23 of the suction tube 2. An inner circumferential surface 13s of the suction hole 13 is located further inward than an inner circumferential surface 2s of the suction tube 2 in a radial direction R.

The distal end opening 13a of the suction hole 13 is arranged apart from the first opening 12a of the through-hole 12 in the radial direction R. The three distal end openings 13a are evenly arranged in a circumferential direction C with respect to the longitudinal direction A. The distal end opening 13a is formed in a long hole shape in the circumferential direction C.

The suction tube (suction conduit member) 2 is formed in a cylinder-like shape. In addition, the “cylinder-like shape” includes an exact cylindrical shape and an approximately cylindrical shape. The suction tube 2 has a suction conduit 23 that forms at least a part of the suction channel SR. The suction conduit 23 is a conduit penetrating in the longitudinal direction A. The suction tube 2 covers the entire circumference of the outer tube 10 along an outer circumferential surface of at least a part of the distal end portion 1a of the sheath 1 in the circumferential direction C. The suction channel SR in the suction conduit 23 is formed between an outer circumferential surface of at least a part of the distal end portion 1a of the sheath 1 and an inner circumferential surface of the suction tube 2.

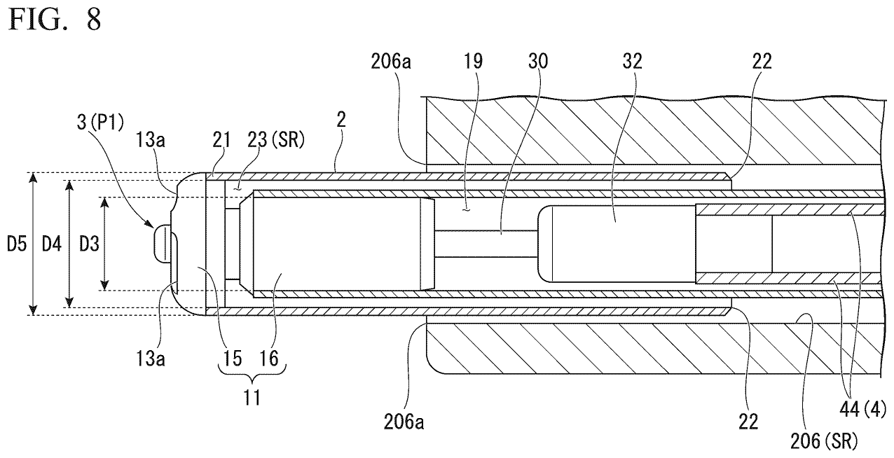

FIG. 8 is a view showing a distal end portion of the treatment instrument 100 at which the distal end of the knife 3 is at the first position P1.

The distal end opening 21 of the suction tube 2 is located in the distal end portion 1a of the sheath 1. The distal end opening 21 of the suction tube 2 is fitted and fixed to the first portion 15. For example, the first portion 15 of the distal end member 11 and the suction tube 2 may be joined by an adhesive.

The first portion 15 of the distal end member 11 is connected to the suction tube 2 at a position on the distal end side A1 and an outward position in the radial direction R rather than at a connection position between the second portion 16 and the outer tube 10.

A fitting diameter D4 between the suction tube 2 and the first portion 15 is larger than a fitting diameter D3 between the outer tube 10 and the second portion 16 (D4>D3). The fitting diameter D4 between the suction tube 2 and the first portion 15 is smaller than a maximum outer diameter D5 of the distal end member 11 (D5>D4).

The proximal end opening 22 of the suction tube 2 is open outside the sheath 1 at a position between the distal end and the proximal end of the sheath 1. As shown in FIG. 5, when the practitioner performs suction treatment or hemostatic treatment using the knife 3 or the like while observing the captured image captured by the imaging portion 203, the distal end opening 21 of the suction tube 2 protrudes from the distal end opening 206a of the channel 206 and a protrusion length L1 in which the distal end of the knife 3 protrudes from the distal end opening 206a of the channel 206 is 5 mm to 10 mm. At this time, the proximal end opening 22 of the suction tube 2 is arranged inside the channel 206 of the endoscope 200. For example, the proximal end opening 22 of the suction tube 2 may be arranged inside the channel 206 in the bending portion 204 of the endoscope 200. A length L2 of the suction tube 2 is shorter than a length of the bending portion 204 of the endoscope 200 along the longitudinal shaft and is, for example, 10 mm to 20 mm. Because the suction tube 2 is prone to buckling if it is excessively long, the length L2 of the suction tube 2 can be, for example, 20 mm or less.

As shown in FIG. 5, the suction conduit 23 communicates with the suction hole 13 via the distal end opening 21 on the distal end side A1. Moreover, the suction conduit 23 communicates with the channel 206 via the proximal end opening 22 on the proximal end side A2. That is, the suction hole 13, the suction conduit 23, and the channel 206 form the suction channel SR. The distal end opening 13a is a suction opening of the suction channel SR. A liquid suctioned from the distal end opening 13a passes through the suction channel SR (the suction hole 13, the suction conduit 23, and the channel 206) and is discharged from the body. From the viewpoint of suction efficiency, a gap between the distal end opening 206a of the channel 206 and the suction tube 2 can be small.

In addition, a connection between the first portion 15 and the suction tube 2 is not limited to the connection based on the above-described fitting. The first portion 15 can be connected to the suction tube 2 so that the suction hole 13 and the suction conduit 23 communicate with each other.

The knife (electrode) 3 is made of metal and is an elongated member. The knife 3 is formed of a material such as, for example, stainless steel. The knife 3 has electrical conductivity and is energized by a high-frequency electric current. The knife 3 has a knife body 30, a flange 31, and a connector 32.

The knife 3 is inserted through the through-hole 12 of the distal end member 11 of the sheath 1 in the longitudinal direction A and can freely protrude or retract from the first opening 12a to the distal end side A1. In addition, the knife 3 may be fixed when the knife 3 cannot advance and retract in a state in which the knife 3 has protruded from the first opening 12a to the distal end side A1.

A central axis O3 of the knife 3 in the longitudinal direction A can coincide with the central axis O1 in the longitudinal direction A of the sheath 1. In addition, “coincidence” includes both an aspect in which they exactly coincide with each other and an aspect in which they substantially coincide with each other.

The knife body 30 is a metallic round rod-like member. An outer diameter of the knife body 30 is smaller than an inner diameter of the through-hole 12. The knife body 30 can advance and retract in the through-hole 12 and the first opening 12a in the longitudinal direction A.

The flange (an enlarged-diameter distal end portion) 31 is a flange-shaped electrically conductive member provided at the distal end of the knife body 30. In the front view as seen in the direction along the longitudinal direction A, the outer circumference of the flange 31 is concentrically formed with the outer circumference of the knife body 30. A length D2 of the flange 31 in the radial direction R perpendicular to the longitudinal direction A is longer than a length D1 of the knife body 30 in the radial direction R. The flange 31 cannot be inserted through the through-hole 12 and the first opening 12a. A plane-like proximal end surface 31b is formed on the proximal end side A2 of the flange 31. In addition, the flange 31 is not limited to a disk-like shape, but may have a triangular shape or a hook shape. Moreover, an insulating resin member may be fixed to the distal end of the knife 3.

As shown in FIG. 5, if the knife 3 is advanced to the sheath 1, the distal end of the connector 32 comes into contact with the distal end member 11 (the second portion 16). When the distal end of the connector 32 comes into contact with the distal end member 11, the distal end of the knife 3 is positioned at the second position P2 that is a position of the most distal end side A1.

As shown in FIG. 8, if the knife 3 is retracted from the sheath 1, the proximal end surface 31b of the flange 31 comes into contact with the distal end member 11 (the first portion 15). When the proximal end surface 31b of the flange 31 comes into contact with the distal end member 11, the distal end of the knife 3 is positioned at the first position P1 that is a position of the most proximal end side A2.

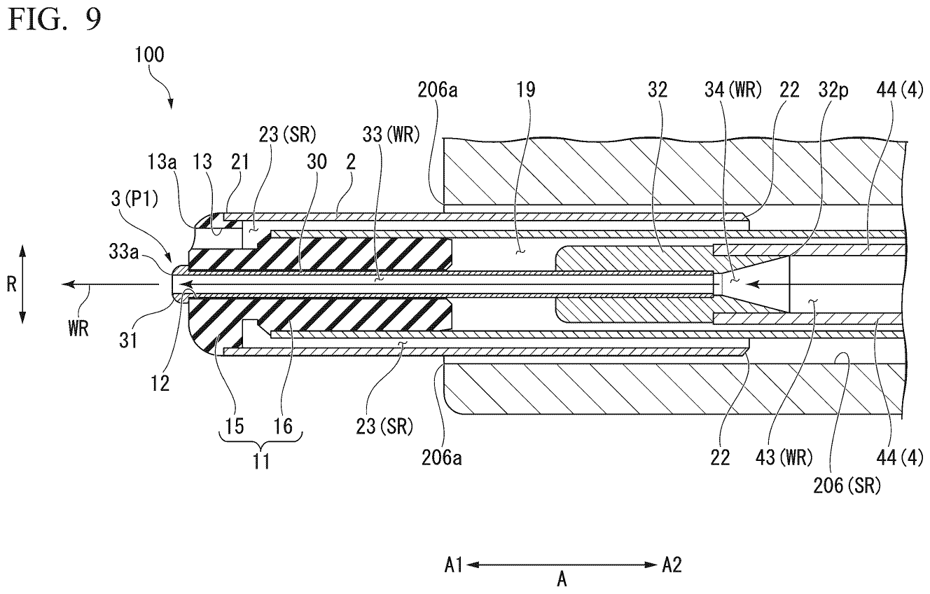

FIG. 9 is a cross-sectional view of the distal end portion of the treatment instrument 100.

As shown in FIG. 9, the knife body 30 and the flange 31 have a first conduit 33 extending in the longitudinal direction A. The first conduit 33 communicates with a distal end opening 33a formed in the flange 31. The distal end opening 33a is an opening formed on the distal end side A1 of the flange 31.

The connector (a connection member) 32 is a cylinder-like member made of metal. In addition, the “cylinder-like” shape includes an exact cylindrical shape and an approximately cylindrical shape. The connector 32 connects the knife body 30 and the manipulation wire 4.

As shown in FIG. 9, when the distal end of the knife 3 is positioned at the first position P1, the proximal end opening 22 of the suction tube 2 is located on the distal end side A1 of a proximal end 32p of the connector 32. Therefore, buckling of the suction tube 2 can be prevented.

The connector 32 has a second conduit 34 extending in the longitudinal direction A. The second conduit 34 communicates with the first conduit 33 and the third conduit 43 formed in the manipulation wire 4. The second conduit 34 is formed in a reduced-diameter taper-like shape from the proximal end side A2 to the distal end side A1.

The manipulation wire 4 is attached to the proximal end of the connector 32. A high-frequency electric current is supplied to the knife 3 via the manipulation wire 4 connected to the manipulation portion 5. When the high-frequency electric current is supplied from the manipulation wire 4 to the knife 3, the knife body 30 and the flange 31 function as active electrodes that output the high-frequency electric current to a biological tissue.

The manipulation wire 4 is a wire inserted through the internal space (the conduit or the lumen) 19 of the outer tube 10 of the sheath 1. The manipulation wire 4 is a wire for manipulating the knife 3. The manipulation wire 4 has a coil shaft 44. A distal end of the manipulation wire 4 is connected to the connector 32 of the knife 3 and the proximal end of the manipulation wire 4 is connected to the slider 52 of the manipulation portion 5. In addition, the manipulation wire 4 may be in other aspects if it is a hollow shaft.

The coil shaft 44 is a hollow coil wire made of metal. The coil shaft 44 is formed of a material such as, for example, stainless steel. A third conduit 43 is formed inside the coil shaft 44. The third conduit 43 is connected to the proximal end of the second conduit 34. The first conduit 33, the second conduit 34, and the third conduit 43 form a water supply channel WR. A fluid supplied from a liquid supply port 54 passes through the water supply channel WR (the third conduit 43, the second conduit 34, or the first conduit 33) and is discharged from the distal end opening 33a.

The coil shaft 44 may have an outer circumference portion covered with a heat shrink tube or the like. By covering the outer circumference portion of the coil shaft 44 with a heat shrink tube, the leakage of the liquid from the third conduit 43 is more difficult.

As shown in FIGS. 1 and 2, the manipulation portion 5 includes a manipulation-portion body 51, a slider 52, a power supply connector 53, and the liquid supply port 54.

The distal end portion of the manipulation-portion body 51 is connected to the proximal end portion 1b of the sheath 1 (the proximal end portion of the outer tube 10). The manipulation-portion body 51 has an internal space through which the manipulation wire 4 can be inserted. The manipulation wire 4 extends to the slider 52 through the internal space 19 of the outer tube 10 and the internal space of the manipulation-portion body 51.

The slider 52 is movably attached to the manipulation-portion body 51 in the longitudinal direction A. The proximal end of the manipulation wire 4 is attached to the slider 52. When the practitioner advances and retracts the slider 52 with respect to the manipulation-portion body 51, the manipulation wire 4 and the knife 3 advance and retract.

The power supply connector 53 is fixed to the slider 52. The power supply connector 53 can be connected to a high-frequency power supply device (not shown) and is connected to the proximal end portion of the manipulation wire 4 via an electrically conductive wire. The power supply connector 53 can supply the high-frequency electric current supplied from the high-frequency power supply device to the knife 3 via the manipulation wire 4. In addition, the power supply connector 53 may be fixed to the manipulation-portion body 51 instead of the slider 52.

The liquid supply port 54 is provided on the slider 52. The liquid supply port 54 is connected to the proximal end of the third conduit 43 via a conduit formed in the slider 52. As shown in FIG. 9, the liquid supplied from the liquid supply port 54 passes through the water supply channel WR (the third conduit 43, the second conduit 34, and the first conduit 33) and is discharged from the distal end opening 33a. In addition, the liquid supply port 54 may be provided in the manipulation-portion body 51 instead of the slider 52.

According to the above configuration, the treatment instrument 100 can perform suction from the distal end of the sheath 1 without significant deterioration in the suction performance of the endoscope 200. That is, the endoscopic treatment system 300 can achieve both the suction efficiency based on channel 206 of the endoscope 200 and the suction efficiency based on the suction tube 2 of the treatment instrument 100.

### [Method Using Endoscopic Treatment System 300]

Next, a procedure using the endoscopic treatment system 300 of the present embodiment (a method using the endoscopic treatment system 300) will be described. Specifically, local injection treatment, incision, and dissection processes for lesion portions in endoscopic therapy such as endoscopic submucosal dissection (ESD) will be described.

As preparatory work, the practitioner identifies a lesion portion by a known method. Specifically, the practitioner inserts the insertion portion 202 of the endoscope 200 into a digestive tract (e.g., an esophagus, a stomach, a duodenum, or a large intestine) and identifies the lesion portion while observing an image obtained by the imaging portion 203 of the endoscope.

### <Insertion Step>

The practitioner inserts the treatment instrument 100 into the channel 206 and causes the distal end portion 1a of the sheath 1 to protrude from the distal end opening 206a of the insertion portion 202.

### <Marking Step>

As shown in FIG. 5, the practitioner relatively retracts the slider 52 of the manipulation portion 5 with respect to the manipulation-portion body 51 and arrange the distal end of the knife 3 at the first position P1. Using the distal end portion of the flange 31 protruding from the distal end surface 14 of the distal end member 11, the biological tissue around the lesion portion is cauterized and marked.

### <Local Injection Step>

As shown in FIG. 5, the practitioner relatively advances the slider 52 of the manipulation portion 5 with respect to the manipulation-portion body 51 and arranges the distal end of the knife 3 at the second position P2. The practitioner supplies water (local injection solution) from the liquid supply port 54 in a state in which the distal end opening 33a of the distal end of the knife 3 is in a submucosal layer. The liquid (local injection liquid) passes through the water supply channel WR (the third conduit 43, the second conduit 34, and the first conduit 33) and is discharged from the distal end opening 33a.

### <Incision and Dissection Step>

Next, the practitioner performs incision and dissection treatment. The practitioner moves the flange 31 while advancing the knife 3 and applying a high-frequency electric current to incise a mucous membrane of the lesion portion. Moreover, the practitioner dissects a submucosal layer of the incised lesion portion while lifting the mucous membrane of the incised lesion portion and exposing the submucosal layer in a state in which the knife 3 advances to apply the high-frequency electric current.

### <Hemostasis Step>

When bleeding occurs during the incision or dissection treatment, the practitioner performs hemostatic treatment. The practitioner supplies water from the liquid supply port 54 and washes an area around a hemostatic point to identify the hemostatic point. Subsequently, the practitioner suctions a liquid that is an electrolyte from a surgical field so that reliable hemostatic treatment is performed. The practitioner presses the suction button 209c and performs suction from the distal end opening 206a through the endoscope 200 (first step). Subsequently, suction is performed from the suction conduit 23 of the treatment instrument 100 (second step). As a result, the liquid suctioned from the distal end opening 13a passes through the suction channel SR (the suction hole 13, the suction conduit 23, and the channel 206) and is discharged from the body. The practitioner may cause the distal end opening of the suction channel SR to be in contact with the liquid so that the suction is efficiently performed.

Subsequently, the practitioner performs hemostasis by applying a high-frequency electric current and cauterizing a bleeding point while pressing the knife body 30 and the flange 31 (third step). At this time, because there is no liquid as an electrolyte in the surgical field, the practitioner can quickly perform hemostatic treatment. The practitioner may cause the knife 3 to be in contact with the liquid so that hemostasis is efficiently performed.

Because the practitioner can perform the hemostatic treatment and the suction treatment using the same treatment instrument 100, it is not necessary to replace the treatment instrument or remove the treatment instrument and then suction blood or the like using the channel 206 of the endoscope 200. In addition, the practitioner may perform the second step and the third step at the same time.

The practitioner will continue the above-described operation (treatment) as necessary and finally remove the lesion portion and end the ESD manipulation.

The treatment instrument 100 according to the present embodiment can perform a suction process from the distal end of the sheath 1 without significant deterioration in the suction performance of the endoscope 200. That is, the endoscopic treatment system 300 can achieve both the suction efficiency based on the channel 206 of the endoscope 200 and the suction efficiency based on the suction tube 2 of the treatment instrument 100. Therefore, the treatment instrument 100 can also perform suction treatment using the suction function of the endoscope 200 in accordance with a situation. Because the treatment instrument 100 can perform the suction treatment and a hemostasis process, the practitioner can immediately identify a hemostatic point and stop bleeding after suctioning and collecting the liquid accumulated in the surgical field.

Although the first embodiment has been described above in detail with reference to the drawings, a specific configuration is not limited to this embodiment and design changes and the like are also included without departing from the scope and spirit of the present invention. Moreover, a configuration in which constituent elements shown in the above-described embodiment and modified examples are appropriately combined can be adopted.

### Modified Example 1

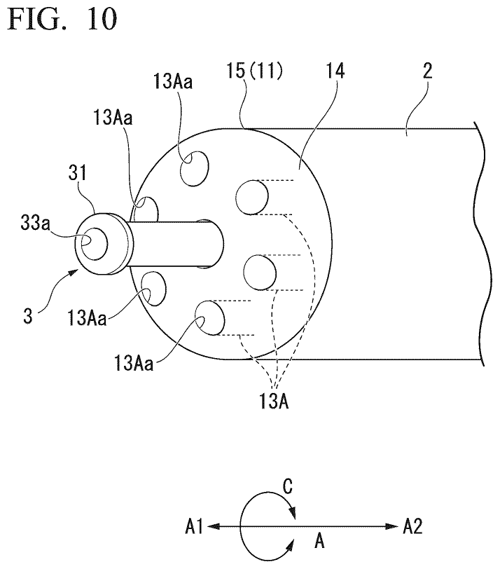

FIG. 10 is a view showing a suction hole 13A, which is a modified example of the suction hole 13.

Six suction holes 13A are formed in the first portion 15 of the distal end member 11. The six suction holes 13 communicate with six distal end openings 13Aa formed in the distal end surface 14 of the distal end member 11. A proximal end of the suction hole 13A communicates with the internal space 19 of the outer tube 10. The six distal end openings 13Aa are evenly arranged in a circumferential direction C with respect to the longitudinal direction A. The distal end opening 13Aa is formed in a circular shape as seen in the longitudinal direction A. Although the suction holes 13A are symmetrically arranged with respect to the central axis of the distal end member 11 in the longitudinal direction A, the suction holes 13A may be arranged asymmetrically with respect to the central axis in the longitudinal direction A.

### Modified Example 2

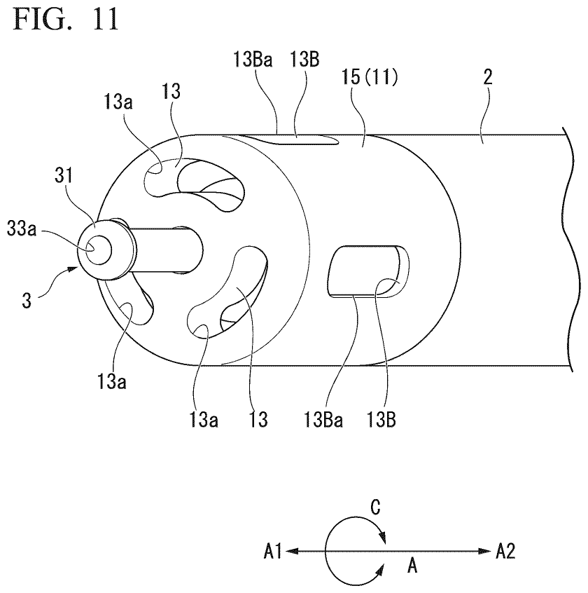

FIG. 11 is a view showing a suction hole 13B, which is a modified example of the suction hole 13.

The suction hole 13B is a side hole formed on the side of the first portion 15 of the distal end member 11. The suction hole 13B communicates with a side opening 13Ba formed on a side surface of the distal end member 11. The suction hole 13B communicates with the internal space 19 of the outer tube 10. The practitioner can suction a liquid accumulated in the surgical field from the suction hole 13B formed on the side. In addition, the distal end member 11 may not have the suction hole 13 and may have only the suction hole 13B.

### Modified Example 3

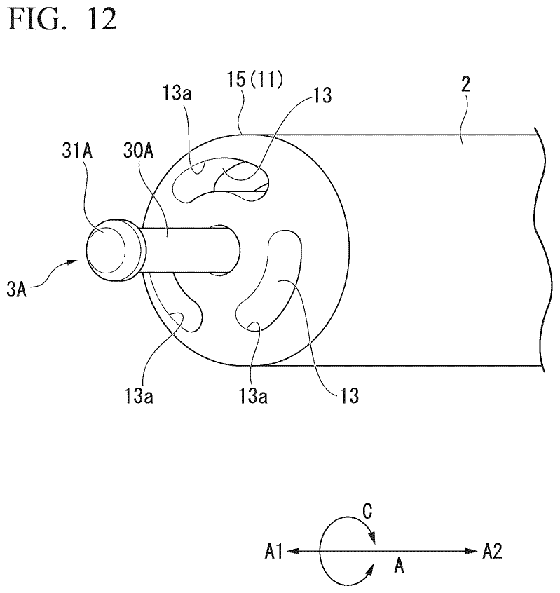

FIG. 12 is a view showing a knife 3A, which is a modified example of the knife 3.

The knife 3A does not have a water supply channel WR and does not have a water supply function. Even if the knife 3A does not have a water supply function, hemostatic treatment and suction treatment can be suitably performed. Because it is not necessary to provide the water supply channel WR in the knife 3A, the connector 32, and the manipulation wire 4, a sheath diameter of the sheath 1 can be thinned and the suction efficiency of the suction channel SR can be improved.

### Modified Example 4

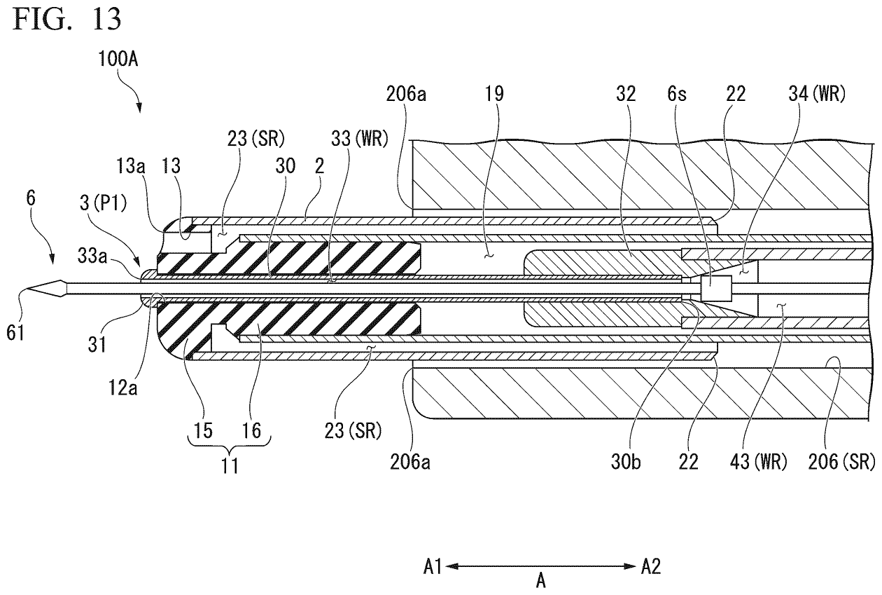

FIG. 13 is a cross-sectional view of a treatment instrument 100A, which is a modified example of the treatment instrument 100.

The treatment instrument 100A further includes a sharp member (rod) 6. The sharp member 6 is a rod-like member formed of a resin material, a metallic material, or the like. The sharp member 6 is inserted through the water supply channel WR of the knife 3 so that the sharp member 6 can advance and retract. The sharp member 6 freely protrudes or retracts from the distal end opening 33a of the knife 3. The sharp portion 61 is formed at the distal end of the sharp member 6. The practitioner punctures a local injection location where a local injection liquid (local injection solution) is injected at the lesion portion with the sharp portion 61 of the sharp member 6, such that the knife 3 can be easily inserted into the local injection location. When the sharp member 6 is retracted to the maximum, the distal end of the sharp member 6 is located between the distal end and the proximal end of the distal end member 11. Alternatively, when the sharp member 6 is retracted to the maximum, the distal end of the sharp member 6 may be located between the proximal end of the distal end member 11 and a proximal end opening 30b of the knife body 30. When the sharp member 6 advances, a stopper 6s provided in the sharp member 6 and the taper-like second conduit 34 come into contact with each other, such that the sharp member 6 is positioned at a position of the most distal end side A1. When the sharp member 6 advances to the maximum, a protrusion length of the sharp member 6 from the distal end of the knife 3 may be shorter than a total length of the suction tube 2. In this case, local injection and suction can be efficiently performed.

### Modified Example 5

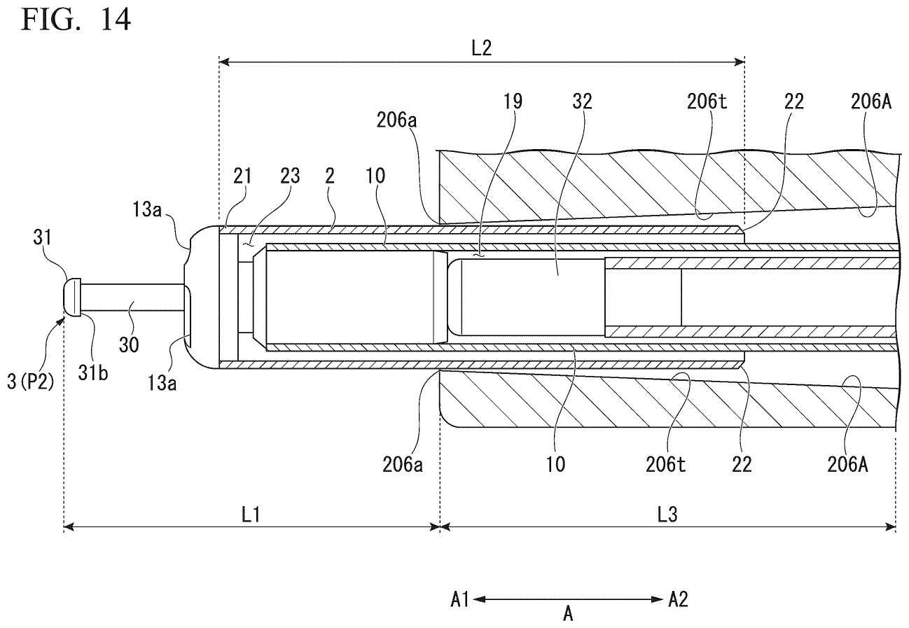

FIG. 14 is a cross-sectional view of a channel 206A, which is a modified example of the channel 206.

The channel 206A has a taper portion 206t formed in a taper-like shape. The taper portion 206t is formed on the distal end portion of the channel 206A and has an inner diameter that narrows toward the distal end opening 206a side. The taper portion 206t can be provided in the sliding range of the suction tube 2. A length L3 of the taper portion 206t in the longitudinal direction A is, for example, 10 mm to 15 mm. Because a gap between the distal end opening 206a of the channel 206A and the suction tube 2 is reduced, the suction efficiency by the channel 206A is improved. In addition, a flap or the like may be provided on the endoscope 200 to fill the gap between the distal end opening 206a of the channel 206A and the suction tube 2.

### Second Embodiment

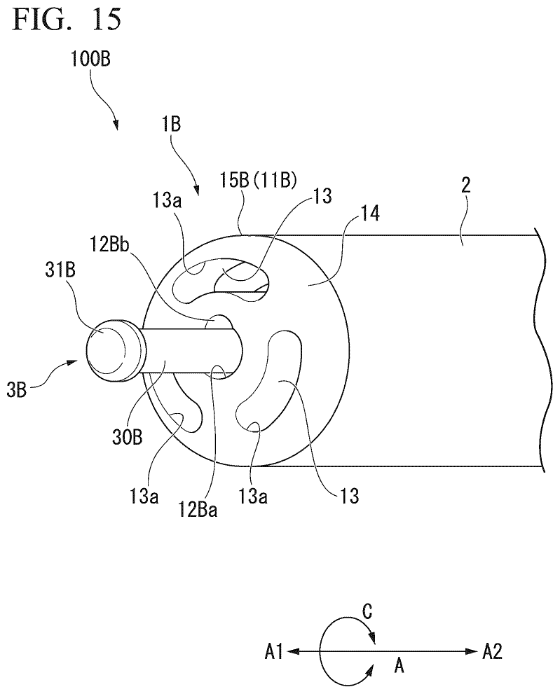

A treatment instrument 100B according to a second embodiment will be described with reference to FIGS. 15 to 17. In the following description, constituent elements identical to those described above will be denoted by the same reference signs and redundant description will be omitted.

FIG. 15 is a perspective view of a distal end portion of the treatment instrument 100B.

The treatment instrument (an endoscopic treatment instrument or a high-frequency treatment instrument) 100B includes a sheath 1B, a suction tube 2, a knife 3B, a manipulation wire 4B, and a manipulation portion 5.

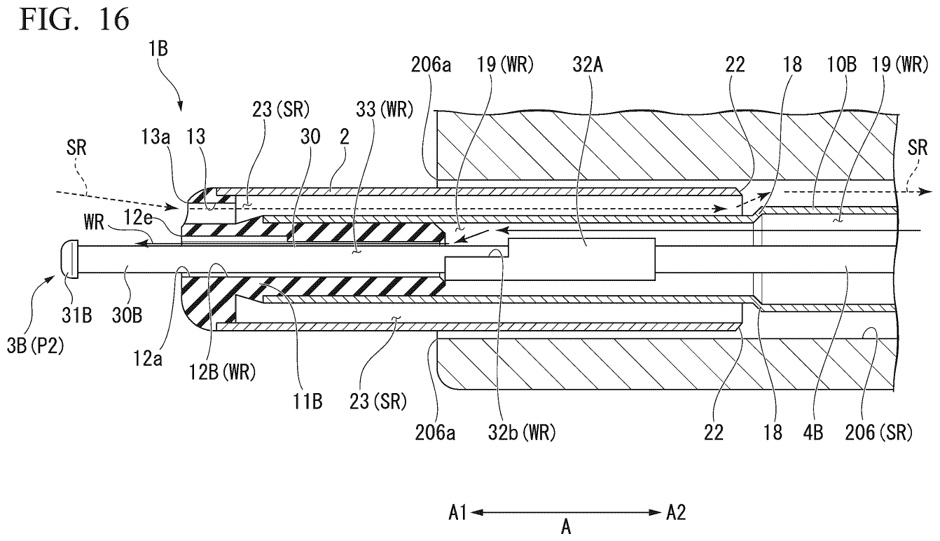

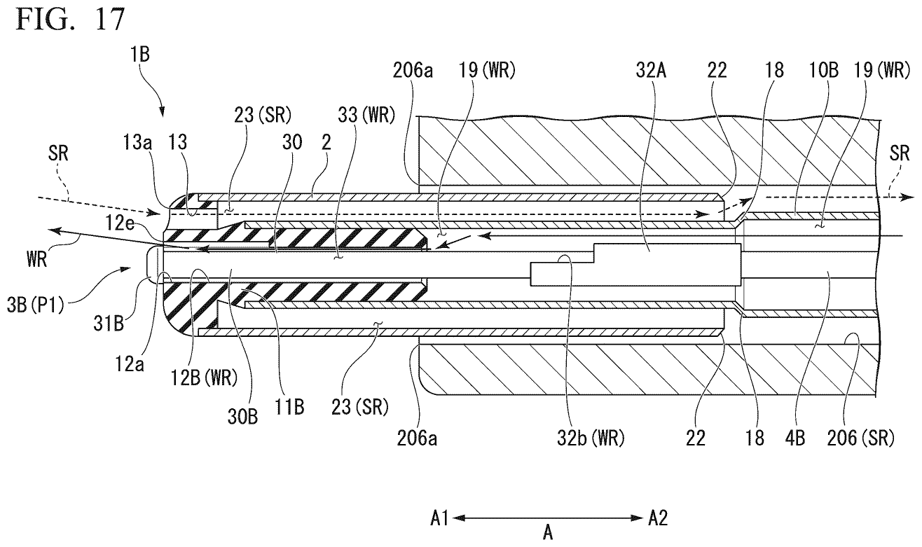

FIG. 16 is a cross-sectional view of the distal end portion of the treatment instrument 100B in which a distal end of the knife 3B is located at a second position P2. FIG. 17 is a cross-sectional view of the distal end portion of the treatment instrument 100B in which the distal end of the knife 3B is located at a first position P1. The sheath 1B includes an outer tube 10B extending in a longitudinal direction A and a distal end member 11B provided at a distal end of an outer tube 10B. The sheath 1B may be formed by integrally molding the outer tube 10B and a distal end member 11B.

The outer tube 10B is a long tubular member having flexibility and insulation. The outer tube 10B is formed, for example, of resin. In the present embodiment, an internal space 19 of the outer tube 10B is a space through which the knife 3B is inserted and is at least a part of a water supply channel WR.

The outer tube 10B has an enlarged diameter portion 18 on a proximal end side A2 of a proximal end opening 22 of the suction tube 2. The enlarged diameter portion 18 is a portion in which the outer diameter increases from a distal end side A1 to a proximal end side A2. In the outer tube 10B, an outer diameter of a portion of the proximal end side A2 of the enlarged diameter portion 18 becomes large. Thereby, because a cross-sectional area of the internal space 19 in the outer tube 10B can be increased, the efficiency of water supply is improved.

The distal end member 11B is formed in a shape similar to that of the distal end member 11 of the first embodiment. A through-hole 12B and a suction hole 13 are formed in the distal end member 11B.

The through-hole 12B is provided in the distal end member 11B and is a hole passing through the distal end member 11B in the longitudinal direction A. The distal end of the through-hole 12B communicates with a first opening 12a and a water supply groove 12e formed in the distal end surface 14 of the distal end member 11B. The first opening 12a and the water supply groove 12e are connected. In addition, the first opening 12a and the water supply groove 12e may not be connected. The proximal end of the through-hole 12B communicates with the internal space 19 of the outer tube 10B. In the present embodiment, the through-hole 12B forms at least a part of the water supply channel WR. A liquid inserted through the through-hole 12B is released from at least the water supply groove 12e.

The knife (an electrode) 3B includes a knife body 30B, a flange 31B, and a connector 32B. The knife 3B is inserted through the through-hole 12B of the distal end member 11B of the sheath 1B in the longitudinal direction A and freely protrudes and retracts from the first opening 12a to the distal end side A1.

The knife body 30B is a metallic round rod-like member. An outer diameter of the knife body 30B is smaller than an inner diameter of the through-hole 12B. The knife body 30B can advance and retract in the through-hole 12B and the first opening 12a in the longitudinal direction A. The knife body 30B cannot be inserted through the water supply groove 12e.

The flange (an enlarged-diameter distal end portion) 31B is a disk-like, electrically conductive member provided at the distal end of the knife body 30A. The flange 31B cannot be inserted through the through-hole 12B, the first opening 12a, and the water supply groove 12e.

The knife body 30B and the flange 31B do not have a first conduit 33 extending in the longitudinal direction A.

The connector (a connection member) 32B further has a notch 32b that forms at least a part of the water supply channel WR on the outer circumferential surface compared with the connector 32 of the first embodiment. When the knife 3B is arranged at the first position P1, a space formed by the notch 32b communicates with the through-hole 12B to form the water supply channel WR.

The manipulation wire 4B is a wire inserted through the internal space (a conduit or a lumen) 19 of the outer tube 10 of the sheath 1B. The manipulation wire 4B is, for example, a solid wire. The manipulation wire 4B does not have a third conduit 43.

A liquid supply port 54 is connected to the proximal end of the internal space 19 of the outer tube 10 via a conduit formed in a slider 52. As shown in FIG. 16, the liquid supplied from the liquid supply port 54 passes through the water supply channel WR (the internal space 19 and the through-hole 12B) and is discharged from the water supply groove 12e. Even when the knife 3B is arranged at the first position P1, the water supply groove 12e is not blocked by the flange 31B and the liquid supplied to the through-hole 12B is discharged from the water supply groove 12e.

Like the treatment instrument 100 of the first embodiment, the treatment instrument 100B according to the present embodiment can perform a suction process from the distal end of the sheath 1B without significant deterioration in the suction performance of the endoscope 200. That is, the suction efficiency based on the channel 206 of the endoscope 200 and the suction efficiency based on the suction tube 2 of the treatment instrument 100B can be achieved. Therefore, the treatment instrument 100B can also perform suction treatment using the suction function of the endoscope 200 in accordance with a situation. Because the treatment instrument 100B can perform suction treatment and a hemostasis process, the practitioner can identify a hemostatic point and stop bleeding immediately after suctioning and collecting the liquid accumulated in the surgical field.

Although the second embodiment has been described above in detail with reference to the drawings, a specific configuration is not limited to this embodiment and design changes and the like are also included without departing from the scope and spirit of the present invention. Moreover, a configuration in which constituent elements shown in the above-described embodiment and modified examples are appropriately combined can be adopted.

### Third Embodiment

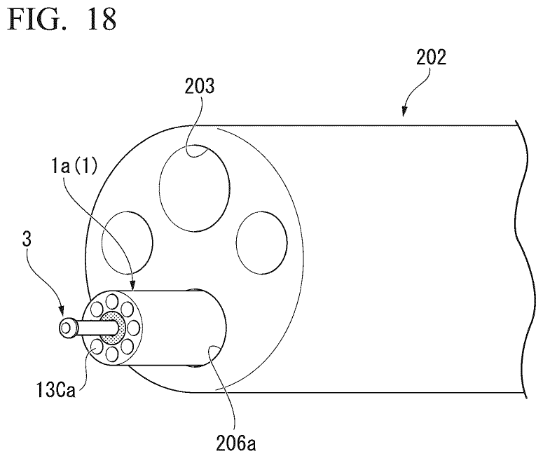

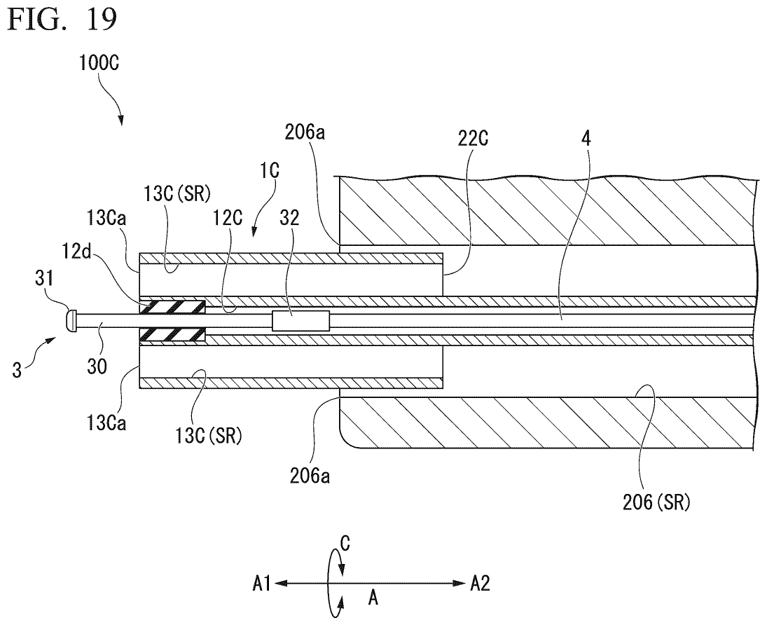

A treatment instrument 100C according to a third embodiment will be described with reference to FIGS. 18 to 19. In the following description, constituent elements identical to those described above will be denoted by the same reference signs and redundant description will be omitted.

FIG. 18 is a perspective view of a distal end portion of the treatment instrument 100C. FIG. 19 is a cross-sectional view of the distal end portion of the treatment instrument 100C. The treatment instrument (an endoscopic treatment instrument or a high-frequency treatment instrument) 100C includes a sheath 1C, a knife 3, a manipulation wire 4, and a manipulation portion 5.

The sheath 1C is a long tubular member extending from a distal end portion 1a to a proximal end portion 1b in a longitudinal direction A along a longitudinal shaft. The sheath 1 can be inserted into a channel 206 of an endoscope 200 and can advance and retract in the channel 206. The sheath 1C is a multi-lumen tube. The sheath 1C includes a knife conduit 12C through which a knife can be inserted to advance and retract and a suction conduit 13C that is at least a part of a suction channel SR.

A distal end opening 13Ca of the suction conduit 13C is located in a distal end portion of the sheath 1C. A proximal end opening 22C of the suction conduit 13C is open outside the sheath 1C at a position between the distal end and the proximal end of the sheath 1C. The proximal end opening 22C of the suction conduit 13C is formed by cutting off at least one lumen of the multi-lumen tube.

When the practitioner performs suction treatment or hemostatic treatment using the knife 3 or the like while observing a captured image captured by an imaging portion 203, the distal end opening 13Ca of the suction conduit 13C protrudes from the distal end opening 206a and a protrusion length L1 in which the distal end of the knife 3 protrudes from the distal end opening 206a of the channel 206 is 5 mm to 10 mm. At this time, a proximal end opening 22C of the suction conduit 13C is arranged inside the channel 206 in the bending portion 204 of the endoscope 200.

As in the first embodiment, a positioning member (a distal end member) 12d for positioning the knife 3 at a first position P1 and a second position P2 is provided at the distal end of the knife conduit 12C.

A connector (a connection member) 32 connects a knife body 30 and a manipulation wire 4 as in the first embodiment. When the knife 3 advances with respect to the sheath 1C, a distal end of a connector 32 and a positioning member 12d come into contact with each other. By causing the distal end of the connector 32 and the positioning member 12d to come into contact with each other, the distal end of the knife 3 is positioned at the second position P2 that is a position of the most distal end side A1. When the knife 3 is retracted with respect to the sheath 1C, a proximal end surface 31b of a flange 31 comes into contact with the positioning member 12d. By causing the proximal end surface 31b of the flange 31 and the positioning member 12d to come into contact with each other, the distal end of the knife 3 is positioned at the first position P1 that is a position of the most proximal end side A2.

Like the treatment instrument 100 of the first embodiment, the treatment instrument 100C according to the present embodiment can perform a suction process from the distal end of the sheath 1C without significant deterioration in the suction performance of the endoscope 200. That is, both the suction efficiency based on the channel 206 of the endoscope 200 and the suction efficiency based on the suction conduit 13C of the treatment instrument 100C can be achieved. Therefore, the treatment instrument 100C can also perform suction treatment using the suction function of the endoscope 200 in accordance with a situation. Because the treatment instrument 100C can perform suction treatment and a hemostasis process, the practitioner can identify a hemostatic point and stop bleeding immediately after suctioning and collecting the liquid accumulated in the surgical field.

Although the third embodiment has been described above in detail with reference to the drawings, a specific configuration is not limited to this embodiment and design changes and the like are also included without departing from the scope and spirit of the present invention. Moreover, a configuration in which constituent elements shown in the above-described embodiment and modified examples are appropriately combined can be adopted.

The present disclosure can be applied to an endoscopic treatment instrument and the like having a hemostatic function.

What is claimed is:

1. An endoscopic treatment instrument comprising:
a sheath including a first tube and a distal end member, the distal end member fixed to a distal end of the first tube, the distal end member including a suction hole;
an electrode protruding from a distal end of the sheath;
a second tube fixed to the distal end member; and
a conduit located between an inner surface of the second tube and an outer surface of the first tube, the conduit communicating with the suction hole,
wherein a proximal end of the conduit is located between a distal end and a proximal end of the first tube.

2. The endoscopic treatment instrument according to claim 1,
wherein the second tube covers the outer surface of the first tube.

3. The endoscopic treatment instrument according to claim 1,
wherein the distal end member includes an insulator having the suction hole.

4. The endoscopic treatment instrument according to claim 1,
wherein the suction hole extends in a longitudinal direction of the sheath.

5. The endoscopic treatment instrument according to claim 1, wherein an outer diameter of the first tube is smaller than an outer diameter of the second tube, and the outer diameter of the first tube is larger than an outer diameter of the electrode.

6. The endoscopic treatment instrument according to claim 1,
wherein the distal end member includes:
an insulator fixed to a distal end of the second tube, the insulator including the suction hole; and
a third tube connecting the first tube to the insulator.

7. The endoscopic treatment instrument according to claim 1,
wherein a length from a distal end of the distal end member to a proximal end of the distal end member is 10 mm to 20 mm.

8. The endoscopic treatment instrument according to claim 1, further comprising:
a wire configured to advance and retract in a longitudinal direction of the sheath, the wire is located in the first tube; and
a connector connecting the electrode to the wire.

9. The endoscopic treatment instrument according to claim 8,
wherein a distal end portion of the electrode has a flange, and
wherein the proximal end of the conduit is located proximally relative to a proximal end of the connector when the flange abuts the distal end member.

10. A treatment system comprising:
an endoscope including a channel; and
a treatment instrument configured to be inserted into the channel, the treatment instrument including a tube,
wherein the tube includes a conduit, and
wherein the conduit including a proximal end opening, the proximal end opening is located in the channel.

11. The treatment system according to claim 10, wherein the conduit includes a distal end opening, when the distal end opening is protruded from the channel, the proximal end opening is located in the channel.

12. The treatment system according to claim 10, wherein a length from a distal end of the tube to a proximal end of the tube is shorter than a length of a bending portion included in the endoscope along a longitudinal axis of the endoscope.

13. The treatment system according to claim 10, wherein, the conduit includes a distal end opening, when the distal end opening is protruded from the channel, the proximal end opening communicates with the channel.

14. The treatment system according to claim 10, wherein the channel includes a taper portion narrowing toward a distal end of the channel.

15. A hemostatic method comprising:
suctioning blood in a channel included in an endoscope;
suctioning the blood in a conduit included in a high-frequency treatment instrument; and
abutting an electrode included in the high-frequency treatment instrument to a tissue with an electric current flowing through the electrode.

16. The hemostatic method according to claim 15,
wherein the suctioning the blood in the conduit includes abutting the conduit to the blood, and
wherein the abutting the electrode includes abutting the electrode to the blood.

17. The hemostatic method according to claim 15, wherein the suctioning blood in the channel is performed in a state in which the high-frequency treatment instrument is located in the channel.

18. The hemostatic method according to claim 15, wherein the suctioning the blood in the conduit and the abutting the electrode are simultaneously performed.

19. The hemostatic method according to claim 15,
wherein the suctioning the blood in the conduit is performed in a state in which a proximal end opening of the conduit is located in the channel, the high-frequency treatment instrument includes a tube, the tube includes the conduit, the conduit includes the proximal end opening.

20. The hemostatic method according to claim 15, wherein, in the suctioning the blood in the conduit, a proximal end opening is included in the conduit, the proximal end opening is located in the channel.
